(19) United States
(12) Patent Application Publication
(10) Pub. No.: US 2017/0065266 A1
(43) Pub. Date: Mar. 9, 2017

(54) SURGICAL RETRACTOR DEVICE AND METHODS AND USES THEREOF

(71) Applicant: Landanger, Chaumont Cedex (FR)

(72) Inventor: Benoit Landanger, Ruffey Les Echirey (FR)

(21) Appl. No.: 14/844,628

(22) Filed: Sep. 3, 2015

(51) Int. Cl. A61B 17/02

(52) U.S. Cl. CPC A61B 17/02; A61B 2017/0237

(57) ABSTRACT

A surgical retractor assembly, method, and uses thereof are provided. The surgical retractor assembly comprises a retractor support comprising a flexible tissue retractor blade and a member for contacting and flexing or bending the flexible tissue retractor blade.

### FIELD

The present disclosure relates to a surgical tissue retractor device, and related methods and uses.

### BACKGROUND

In this specification where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

Surgical retractor devices are known in the art, but do not provide optimal support. Thus, there is a need for surgical retractor devices that provide ideal retraction during surgical procedures.

### SUMMARY

In an exemplary embodiment, a surgical retractor assembly comprises a retractor support comprising a flexible retractor blade and at least one member for contacting and flexing and/or bending the flexible and/or rotatable tissue retractor blade.

The exemplary embodiments herein comprise a method of retracting tissue during surgery comprising:

(i) providing a surgical retractor assembly comprising:
(a) a retractor support comprising a flexible and/or rotatable tissue retractor blade comprising at least one edge region and a connection region that is located at a fixed distance from the edge region, wherein the connection region is connected to the retractor support, and the edge region is not connected to the retractor support;
(b) at least one member for contacting and flexing and/or bending the flexible and/or rotatable tissue retractor blade;
wherein when the flexible tissue retractor blade is flexed and/or bent by the member (b), the blade can flex and/or bend so that one portion of the blade can never be attached to or connected to another portion of the blade, even when the blade is flexed and/or bent;

(ii) inserting the flexible and/or rotatable tissue retractor blade in a manner parallel or substantially parallel to the y axis of the retractor support;

(iii) flexing and/or bending the tissue retractor blade with the member (b);

(iv) rotating the tissue blade to become parallel or substantially parallel to the x axis of the retractor support while simultaneously or after flexing and/or bending the tissue retractor blade with the member (b); and

(v) releasing the flexible and/or rotatable tissue retractor blade thereby contacting and retracting tissue in or around the body region or cavity.

### BRIEF DESCRIPTION OF THE DRAWINGS

These and other features of this invention will now be described with reference to the drawings of certain embodiments which are intended to illustrate and not to limit the invention.

FIG. 1 shows a surgical retractor assembly.

FIG. 2 shows a side view of an embodiment of a tissue retractor blade.

FIG. 3 shows an alternative embodiment of a tissue retractor blade.

FIG. 4 shows a retractor support.

FIG. 5 shows a member for contacting and flexing a flexible tissue retractor blade.

FIG. 6 shows the various positions of an embodiment of the retractor blade during an exemplary method of retracting tissue.

### DETAILED DESCRIPTION

Further aspects, features and advantages will become apparent from the detailed description which follows.

The assemblies and methods described herein are intended to encompass assemblies and methods, which consist of, consist essentially of, as well as comprise, the various constituents identified herein (in various combinations and configurations), unless explicitly indicated to the contrary.

Any numbers expressing quantities, measurements, and so forth used in the specification are to be understood as being modified in all instances by the term “about.” Notwithstanding that the numerical ranges and parameters setting forth, the broad scope of the subject matter presented herein are approximations, the numerical values set forth are indicated as precisely as possible. Any numerical value, however, may inherently contain certain errors or inaccuracies as evident from the standard deviation found in their respective measurement techniques. None of the features recited herein should be interpreted as invoking 35 U.S.C. §112(f), unless the term “means” is explicitly used.

As noted above, in its broader aspects, exemplary embodiments disclosed herein are directed to a surgical retractor assembly and related methods comprising, consisting of, or consisting essentially of a one or more of a retractor support, a flexible and/or rotatable tissue retractor blade, and a member for contracting and flexing and/or bending the tissue retractor blade. The surgical retractor assembly is suitable for retracting any tissue during surgical procedures known to those having ordinary skill in the art. As a non-limiting example, the surgical retractor assembly can be an atrial retractor assembly useful, for example, in improving exposure of the mitral valve by retracting or moving heart tissue. As another non-limiting example, the surgical retractor assembly can be an aortic retractor assembly.

Unless otherwise indicated, the terms “attach,” “attached,” “attachable,” “attachment,” “connect,” “connected,” “connector,” and “connectable” means to fasten, affix, or adhere to in some permanent or semi-permanent manner for some period of time.

Unless otherwise indicated, the terms “contact” and “contacted” means the act or state of touching, a touching or meeting, and/or immediate proximity or association.

Accordingly, a person having ordinary skill in the art understands that it is possible, within the scope of the exemplary embodiments, for a first element of the assembly to “contact” (touch or meet) a second element of the assembly without being “attached” or “connected” (fastened or affixed) to the second element of the assembly.

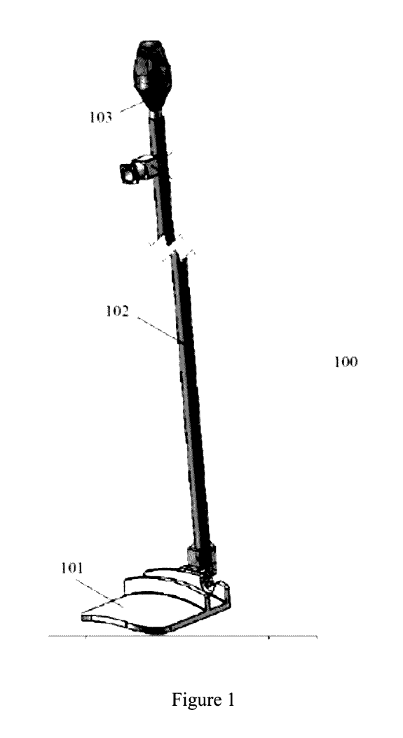

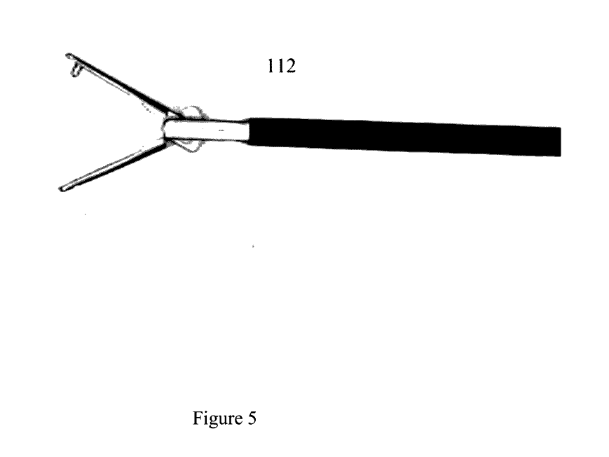

Referring to the Figures, like numerals can indicate corresponding parts throughout the several views the exemplary surgical retractor assembly shown in FIGS. 1-6.

As shown in FIG. 1, the surgical retractor assembly (100) comprises a retractor support (101) and a handle (102) connected to the retractor support (101). In a preferred embodiment, the handle (102) further comprises a grip (103).

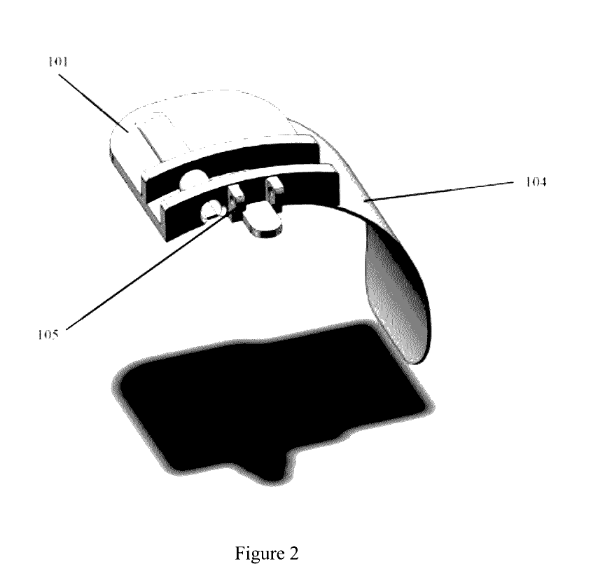

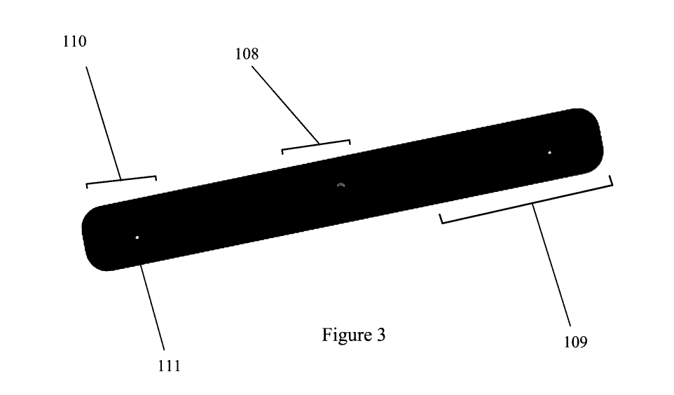

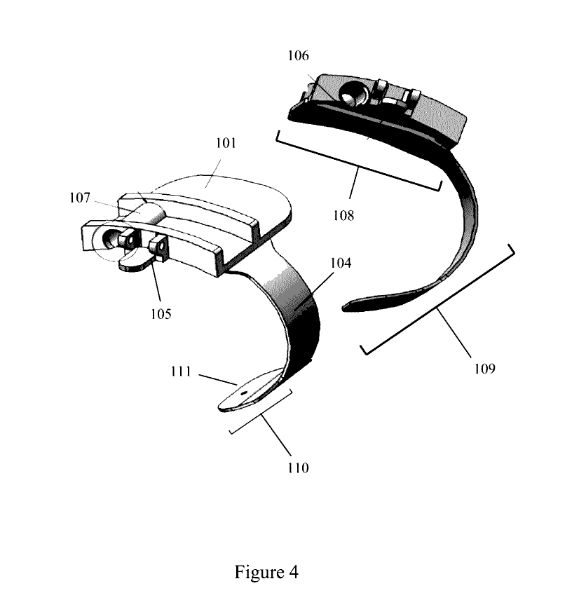

As shown in FIGS. 2-4, in an exemplary embodiment, the retractor support (101) further comprises a handle engagement means (105) and a flexible and/or rotatable tissue retractor blade (104). The flexible and/or rotatable tissue retractor blade (104) comprises at least one edge region (109) and a connection region (108) that is located at a fixed distance from the edge region. The connection region is connected to the retractor support. The edge region (109) is not connected to the retractor support (101). As shown in FIG. 4, in an exemplary embodiment, the connection region (108) is at a fixed distance that is opposite the edge region (109). As shown in FIG. 3, in another exemplary embodiment, the connection region (108) is located at a fixed distance in between the two edge regions (109).

The flexible and/or rotatable tissue retractor blade (104) attaches to the retractor support (101) by methods and means known to those having ordinary skill in the art. In a preferred embodiment, the tissue retractor blade (104) comprises a connector (106) that attaches to the retractor support (101) at connector (107). In an exemplary embodiment, the blade connector (106) is a male connector, and the retractor support connector (107) is a female connector. As a non-limiting example, the connector (106) can be a spherical pin. In another exemplary embodiment, the connector (107) is attached to the retractor support (101) by laser welding, metal injection, or polymer molding.

In an exemplary embodiment, the retractor support (101) has a concave or otherwise curved or bent shape. In an exemplary embodiment, the at least one edge region (109) of the blade (104) comprises a focus region (110). In yet another embodiment, the at least one edge region (109) of the tissue retractor blade (104) comprises an opening (111). The tissue retractor blade (104) comprises materials known to those having ordinary skill in the art. In a preferred embodiment, the tissue retractor blade (104) comprises a polymer. As a non-limiting example, the tissue retractor blade could comprise polyether ether ketone (PEEK), polyamide (PA66), acetal copolymer (POM C), polypropylene (PP), acrylonitrile butadiene styrene (ABS), and mixtures thereof.

In an exemplary embodiment, the surgical retractor assembly further comprises a member for contacting and flexing and/or bending the tissue retractor blade (104). The tissue retractor blade (104) is flexed and/or bent by any means or methods known to those having ordinary skill in the art. In an exemplary embodiment, the member for contacting and flexing and/or bending the tissue retractor blade (104) is a pair of forceps (112). In other embodiments, the member can be any device, element, or means capable of flexing and/or bending the blade (104). In yet another preferred embodiment, the member for contacting and flexing and/or bending the tissue retractor blade (104) comprises a hook for contacting and flexing and/or bending the tissue retractor blade (104) at the opening (111) of the edge region(s) (109).

In a preferred embodiment, when the flexible and/or rotatable tissue retractor blade (104) is flexed and/or bent by the member for contacting and flexing and/or bending the retractor blade (104), the blade (104) can flex and/or bend so that one portion of the blade (104) moves closer to or makes contact with another portion of the blade (104). During the flexing and/or bending, one portion of the blade (104) can never attach to or connect with another portion of the blade (104), even when the blade (104) is flexed and/or bent. Thus, the connection region (108) of the blade (104) will never attaches to or is connected with an edge region (109) of the blade (104).

Exemplary embodiments also include a method of retracting or moving tissue during surgery. In an exemplary embodiment, the method comprises the steps of:

(i) providing a surgical retractor assembly (100) comprising:
(a) a retractor support (101) comprising a flexible and/or rotatable tissue retractor blade (104) comprising at least one edge region (109) and a connection region (108) that is located at a fixed distance from the edge region (109), wherein the connection region (108) is connected to the retractor support (101), and the edge region (109) is not connected to the retractor support (101);
(b) at least one member for contacting and flexing and/or bending the flexible and/or rotatable tissue retractor blade (104);
wherein when the flexible and/or rotatable tissue retractor blade (104) is flexed and/or bent by the member (b), the blade (104) can flex and/or bend so that one portion of the blade (104) can never be attached to or connected to another portion of the blade (104), even when the blade (104) is flexed and/or bent; and

(ii) inserting the flexible and/or rotatable tissue retractor blade (104) in a manner parallel or substantially parallel to the y axis of the retractor support (101);

(iii) flexing and/or bending the tissue retractor blade (104) with the member (b);

(iv) rotating the tissue retractor blade (104) to become parallel or substantially parallel to the x axis of the retractor support (101) while simultaneously flexing and/or bending or after flexing and/or bending the tissue retractor blade (104) with the member (b); and

(v) releasing the flexible and/or rotatable tissue retractor blade (104) thereby contacting and retracting or moving tissue in or around the body region or cavity.

The surgical retractor assembly (100) can be any surgical retractor assembly known to those having ordinary skill in the art. As a non-limiting example, the surgical retractor assembly (100) is an atrial retractor assembly for use in, for example, atrial wall tissue retraction. Such an atrial retractor can improve or enhance exposure of the mitral valve by retracting or moving heart tissue. As another, non-limiting example, the surgical retractor assembly can be an aortic retractor assembly.

Exemplary embodiments herein also comprise a method of retracting tissue during surgery comprising steps (i) through (v) recited above, wherein steps (iii) and (iv) occur simultaneously.

Exemplary embodiments herein also comprise a method of retracting tissue during surgery comprising two members (b) for flexing and/or bending the tissue retractor blade (104) followed by the necessary steps to place the tissue retractor blade in a desired surgical position. In an exemplary method, the tissue retractor blade (104) is retracted and/or bent with two pairs of forceps (112).

In an exemplary method, the retractor support (101) further comprises a handle engagement means (105) and a flexible and/or rotatable tissue retractor blade (104). The flexible and/or rotatable tissue retractor blade (104) comprises at least one edge region (109) and a connection region (108) that is located at a fixed distance from the edge region (109). The connection region is connected to the retractor support. The edge region (109) is not connected to the retractor support (101). In an exemplary embodiment, the connection region (108) is at a fixed distance that is opposite the edge region (109). In another exemplary embodiment, the connection region (108) is located at a fixed distance in between the two edge regions (109).

In an exemplary method, the retractor support (101) has a concave or otherwise curved or bent shape. In an exemplary embodiment, the at least one edge region (109) of the blade (104) comprises a focus region (110). In yet another embodiment, the at least one edge region (109) of the tissue retractor blade (104) comprises an opening (111). The tissue retractor blade (104) comprises materials known to those having ordinary skill in the art. In a preferred embodiment, the tissue retractor blade (104) comprises a polymer. As a non-limiting example, the tissue retractor blade could comprise polyether ether ketone (PEEK), polyamide (PA66), acetal copolymer (POM C), polypropylene (PP), acrylonitrile butadiene styrene (ABS), and mixtures thereof.

The flexible and/or rotatable tissue retractor blade (104) attaches to the retractor support (101) by methods and means known to those having ordinary skill in the art. In an exemplary method, the flexible and/or rotatable tissue retractor blade (104) comprises a connector (106) that attaches to the retractor support (101) at connector (107). In an exemplary method, the blade connector (106) is a male connector, and the retractor support connector (107) is a female connector. As a non-limiting example, the blade connector (106) can be a spherical pin. In another exemplary method, the connector (107) is attached to the retractor support (101) by laser welding, metal injection molding, or by polymer molding.

In an exemplary method, the member (b) is a pair of forceps (112). In another exemplary method, the member (b) comprises a hook for contacting and flexing and/or bending the tissue retractor blade (104) at the opening (111) of the edge region(s) (109).

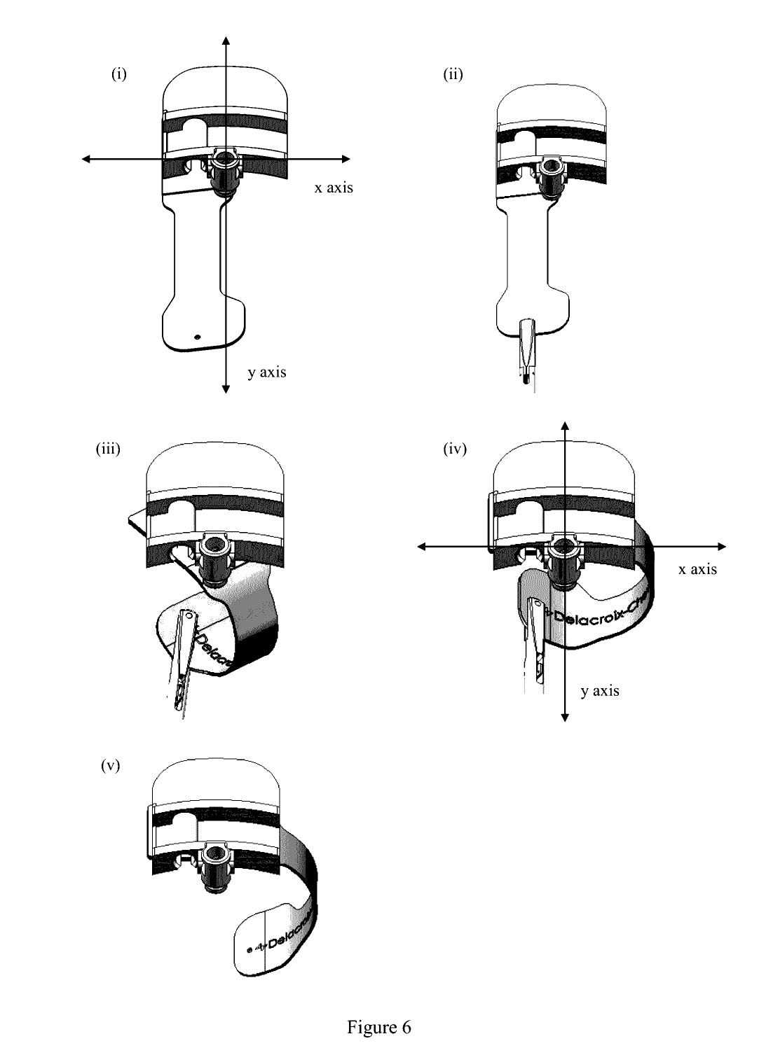

The inserting step (ii) is accomplished by methods and means known to those having ordinary skill in the art. In an exemplary method, the inserting step (ii) is adjusted accordingly to insert the retractor blade (104) into a small body region or cavity. For example, the assembly is inserted with the retractor blade parallel or substantially parallel to the y axis of the retractor support, as depicted in FIG. 6. This method of insertion allows for the blade to enter into small openings, such as, for example, atrial wall tissue. Such small opening insertion capability allows for less invasive surgical procedures.

The flexing and/or bending step (iii) and the rotating step (iv) are accomplished by methods and means known to those having ordinary skill in the art. In an exemplary embodiment, the at least one edge region is seized with a pair of dedicated forceps (112). In another exemplary embodiment, the forceps (112) contact the tissue retractor blade (104) at opening (111) to allow superior control of the blade during flexing and/or rotating. This superior control makes movement of the tissue retractor blade (104) safer and easier to control. In an exemplary embodiment, the flexing and/or bending step (iii) and the rotating step (iv) are conducted simultaneously or in sequence.

The releasing step (v) is accomplished by methods and means known to those having ordinary skill in the art. In an exemplary method, at step (v), the flexible and/or rotatable tissue retractor blade (104) contacts or attaches to a tissue such that a force or flexion is applied to the tissue retractor blade (104). In an exemplary method, the tissue retractor blade (104) is released by disengagement of the retractor blade (104) and the member (b).

In an exemplary embodiment, the method further comprises step (vi) of flexing and/or bending the tissue retractor blade (104) in the body region or cavity and then removing the blade (104) from the body region or cavity.

It is to be understood that the embodiments and methods described herein are merely illustrative. Reference herein to details of the illustrated embodiments is not intended to limit the scope of the claims.

What is claimed is:

1. A surgical retractor assembly comprising:
(a) a retractor support comprising a flexible and/or rotatable tissue retractor blade comprising at least one edge region and a connection region that is located at a fixed distance from the edge region, wherein the connection region is connected to the retractor support, and the edge region is not connected to the retractor support;
(b) at least one member for contacting and flexing and/or bending the flexible and/or rotatable tissue retractor blade,
wherein when the flexible and/or rotatable tissue retractor blade is flexed and/or bent by the member (b), the blade can flex and/or bend so that one portion of the blade can never be attached to or connected to another portion of the blade, even when the blade is flexed and/or bent; and
wherein in an initial insertion state, the tissue retractor blade is positioned in a manner parallel or substantially parallel to the y axis of the retractor support and when inserted the tissue retractor blade is rotatable and/or bendable to be parallel or substantially parallel to the x axis of the retractor support.

2. The surgical retractor assembly according to claim 1, wherein the connection region is located at a fixed distance that is opposite the edge region.

3. The surgical retractor assembly according to claim 1, wherein the connection region is located at a fixed distance in between two edge regions.

4. The surgical retractor assembly according to claim 1, wherein the retractor support has a concave shape.

5. The surgical retractor assembly according to claim 1, wherein the flexible tissue retractor blade has an elliptical shape.

6. The surgical retractor assembly according to claim 1, wherein the member (b) for contacting and flexing and/or bending the flexible tissue retractor blade is a pair of forceps.

7. The surgical retractor assembly according to claim 1, wherein the at least one edge region comprises an opening.

8. The surgical retractor assembly according to claim 7, wherein the member (b) for contacting and flexing and/or bending the flexible tissue retractor blade comprises a hook for contacting and flexing and/or bending the tissue retractor blade at the opening in the at least one edge region.

9. The surgical retractor assembly according to claim 1, wherein the surgical retractor assembly is an atrial retractor assembly or an aortic retractor assembly.

10. A method of retracting tissue during surgery comprising:
(i) providing a surgical retractable assembly comprising;
(a) a retractor support comprising a flexible and/or rotatable tissue retractor blade comprising at least one edge region and a connection region that is located at a fixed distance from the edge region, wherein the connection region is connected to the retractor support, and the edge region is not connected to the retractor support;
(b) at least one member for contacting and flexing and/or bending the flexible and/or rotatable tissue retractor blade,
wherein when the flexible and/or rotatable tissue retractor blade is flexed and/or bent by the member (b), the blade can flex and/or bend so that one portion of the blade can never be attached to or connected to another portion of the blade, even when the blade is flexed and/or bent; and
(ii) inserting the flexible and/or rotatable tissue retractor blade in a manner parallel or substantially parallel to the y axis of the retractor support;
(iii) flexing and/or bending the tissue retractor blade with the member (b);
(iv) rotating the tissue retractor blade to become parallel or substantially parallel to the x axis of the retractor support while simultaneously or after flexing and/or bending the tissue retractor blade with the member (b); and
(v) releasing the flexible and/or rotatable tissue retractor blade thereby contacting and retracting tissue in or around the body region or cavity.

11. The method according to claim 10, wherein the connection region is located at a fixed distance that is opposite the edge region.

12. The method according to claim 10, wherein the connection region is located at a fixed distance in between two edge regions.

13. The method according to claim 10, wherein the retractor support has a concave shape.

14. The method according to claim 10, wherein the member (b) for contacting and flexing and/or bending the tissue retractor blade is a pair of forceps.

15. The method according to claim 10, wherein the at least one edge region comprises an opening.

16. The method according to claim 15, wherein the member (b) for contacting and flexing and/or bending the tissue retractor blade comprises a hook for contacting and flexing and/or bending the tissue retractor blade at the opening of the at least one edge region.

17. The method according to claim 10, further comprising step (vi) flexing and/or bending the tissue retractor blade in the body region or cavity and then removing the blade from the body region or cavity.

18. The method according to claim 10, wherein the surgical retractor assembly is an atrial retractor assembly or an aortic retractor assembly.
